(19) United States
(12) Patent Application Publication
(10) Pub. No.: US 2005/0025719 A1
(43) Pub. Date: Feb. 3, 2005

(54) Method and apparatus for oral hygiene products with green tea extract

(76) Inventor: Kenneth H. Nussen, Santa Monica, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/682,747

(22) Filed: Oct. 8, 2003

Related U.S. Application Data
(63) Continuation-in-part of application No. 10/177,535, filed on Jun. 21, 2002.

(51) Int. Cl. A61K 9/68; A61K 35/78

(52) U.S. Cl. 424/48; 424/729

(57) ABSTRACT

Oral hygiene products including chewing gum, toothpicks, mouthwash, and dentifrice strips are adapted to deliver active ingredients including green tea extract to the mouth of a consumer. A method including mixing one or more base components with green tea extract (GTE) to form a composition or treatment that is used to form the oral hygiene products.

### CROSS-REFERENCE TO RELATED APPLICATION

The application is a continuation-in-part of co-pending application Ser. No. 10/177,535, filed Jun. 21, 2002, by Kenneth H. Nussen, entitled DENTAL HYGIENE PRODUCTS AND METHODS OF MAKING DENTAL HYGIENE PRODUCTS.

### BACKGROUND

1. Field

Oral hygiene products including toothpicks, dental chewing gum, mouthwash and dentifrice strips.

2. Background

Oral hygiene products include toothpicks, mouthwash, dental chewing gum, and dental strips. Dentifrice deliver cleansing agents and tartar control substances that protect teeth and counteract plaque build up. These products typically use artificial ingredients such as artificial sweeteners, flavorants and coloring.

Toothpicks are small elongated pieces of refined materials such as wood or plastic that are used to remove food lodged between teeth. Toothpicks do not provide any additional protection to teeth or deliver agents to counteract bad breath. Gum is often made of chicle that is sweetened and prepared for recreational use such as chewing. Gum generally does not include agents that protect or clean teeth.

Mouthwash is a liquid used to rinse the mouth of a user in order to combat bad breath and plaque build up. Mouthwash often includes antiseptic agents to kill organism that cause bad breath and plaque.

An oral hygiene product incorporating tea tree oil can provide an anti-bacterial effect that may help to reduce bad breath and retard the development of plaque. Tea tree oil has several drawbacks, however, including its strong medicinal odor and its inadequacy at preventing inflammation of the gums. These factors have contributed to the unavailability of tea tree oil oral hygiene products that are both pleasant to use and have anti-inflammatory properties.

Green tea extract is derived from the leaves of the plant Camellia sinensis and is rich in antioxidants. Green tea extract is commonly available in pill form as a nutritional supplement. Green tea extract has been shown to be effective at fighting cavities, prohibiting the growth of oral bacteria, and reducing inflammation of the gums.

### BRIEF DESCRIPTION OF THE DRAWINGS

The claims are illustrated by way of example and not by way of limitation in the figures of the accompanying drawings in which like references indicate similar elements. It should be noted that different references to “an” or “one” embodiment in this disclosure are not necessarily to the same embodiment, and such references mean at least one.

FIG. 1a is a view of a single strip of packaged gum.

FIG. 1b is a view of multiple strips of packaged gum.

FIG. 1c is a view of blister pack of gum.

FIG. 2a is a view of a single treated toothpick.

FIG. 2b is a view of a packaged set of toothpicks.

FIG. 3 is a view of a container of mouthwash.

FIG. 4a is a view of a dentifrice strip.

FIG. 4b is a view of a dentifrice strip placed on the teeth of a user.

### DETAILED DESCRIPTION

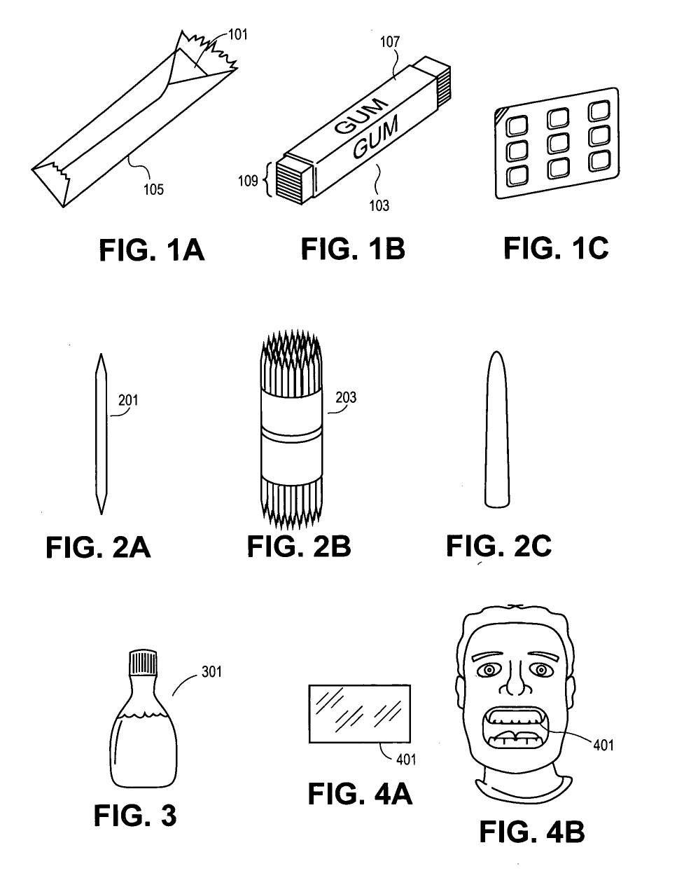

FIG. 1a is an illustration of a piece of chewing gum that has anti-bacterial active ingredients. The strip of gum 101 is contained within foil 105. Foil 105 can be opened by unfolding to expose the strip of gum 101. Gum 101 is then removed from the foil 105 and placed in the mouth of a consumer. Chewing gum 101 is made by combining one or more gum compositions with one or more active ingredients. Suitable gum compositions include, but are not limited to, chicle, jelutong, xylitol, and similar natural and artificial substances. These base gum compositions may be combined with an active ingredient, such as an amount of green tea extract (GTE) that is effective at reducing oral bacteria and fighting cavities. A suitable amount includes, but is not limited to a range of 1.5 to 2.5 percent by weight GTE in a batch. In another embodiment, GTE is combined with tea-tree oil (TTO) in an amount sufficient to mask the odor and taste of the TTO.

Flavoring (e.g., mint, cinnamon, bubble gum, anise, etc.), coloring, and sweeteners may optionally be added to the gum composition. In one embodiment, one or more sweeteners such as saccharin, fructose, sucrose, maltose, and corn syrup are added to the gum composition. The flavoring, coloring and sweeteners may be combined before being added to the gum composition. In one embodiment, mint, cinnamon, bubble gum, anise, or similar flavor is added to the gum composition, for example, in an amount of approximately five percent by weight of the composition.

The gum composition may be formed by hydrating one or more base gum ingredients to create a gum composition. In one embodiment, freeze-dried GTE commercially available from XEL Herbaceuticals of Salt Lake City, Utah is added to the gum composition. In an embodiment where the gum also includes TTO, TTO liquid commercially available from Natural extracts of Australia of Los Angeles, Calif. is added to the gum composition.

The following table is an exemplary composition of the chewing gum composition:

| Item | Dry Weight |
|---|---|
| Xylitol | 44.38-49.05% |
| Maltitol syrup | 2.87-3.17% |
| GTE | 1.79-1.98% |
| Natural Flavor | 1.26-1.39% |
| Gum Arabic | 0.48-0.53% |
| Lecithin | 0.38-0.42% |
| 1. MAG | 0.36-0.40% |
| (Monoammonium | |
| glycyrrhizinate) | |
| 2. Stevia Extract | 0.14-0.16% |
| (Chlorophyll) | 0.02-0.03% |
| Carnauba Wax | 0.01-0.01% |

Note:
1. MAG or
2. Stevia Extract may be used in alternate embodiments

In one embodiment, the gum composition is entirely formed of natural ingredients. Artificial sweetners, colorings and similar substances are not used. Anise, cinnamon, fruit concentrates, mint or similar naturally occurring spices and substances or combinations thereof may be used as flavorants. Anise oil also has anti-bacterial properties. Chlorophyll or similar substances may be used in the composition as a natural coloring. Chlorophyll and other natural coloring substances are derived from vegetables, plants, fruits and similar natural sources. For example, red coloring may be derived from grape skins, orange coloring from beta carotene and gray or white coloring from calcium carbonate. Tapioca starch may be used as natural water binding agent and texturizing agent. In one embodiment, stevia extract, xylitol, malitol, monoammonium glycyerrhizinate, or similar substances may be used as a sweetener. GTE is used in the all natural composition and provides anti-cavity, anti-bacterial, and anti-inflammatory properties.

The gum composition may be heated to promote dissolution of the ingredients and subsequent drying. The gum composition and the active ingredients must be in amounts that are non-toxic and safe for human consumption. In one embodiment, the active ingredients may be combined and optionally hydrated before being added to the gum composition. The resulting gum composition is then transferred to a suitable substrate where it dries to form a thin film. A suitable substrate is nonporous and will not react chemically with the gum composition. As the gum composition dries on the substrate, it changes state from a malleable composition to a relatively stiff film. Drying can be accelerated by exposing the gum composition to heated air or by heating the substrate. When dried, the film is cut into strips of suitable size for consumption and packaged. Representative packaging includes, but is not limited to, a spool, a single use package (FIG. 1a), and a multiple use package (FIG. 1b).

The film is then cut into strips and packaged. The film strips (i.e., sticks of gum 101) are used by consumers in the following manner. A consumer will remove a stick of gum 101 from its packaging and place the stick in their mouth. The stick will become pliable as it comes in contact with saliva, thereby releasing active ingredients such as GTE and TTO and making it suitable for chewing.

FIG. 1b is a view of a multiple stick package of gum 103. The package is comprised of wrapper 107 and individually wrapped sticks 109. Sticks 109 may be removed from wrapper 107 laterally and the gum obtained by removal of the individual foil. FIG. 1c is an alternative packing where the dental chewing gum is formed as small squares and sealed in a blister pack 111.

It is appreciated that in alternative embodiments various other chewing gum types can be formed to provide a desirable substance or substances to a user. For example, gum can be formed that includes one or more vitamins that are necessary for or improve nutrition, or that act as coenzymes and precursors of coenzymes in the regulation of metabolic processes. Representative vitamins include, but are not limited to, Vitamin A, beta kerotene, Vitamin B, Vitamin B6, Vitamin B12, folic acid or folate, Vitamin C, Vitamin D, Vitamin E (including tocopherols) and or tocotrienols. Dental chewing gum may additionally or alternatively include minerals such as calcium, zinc, or similar materials. For example, paste may include an amount of a zinc compound such as zinc gluconate or other zinc compound in an amount sufficient to inhibit symptoms associated with a common cold. In one embodiment, the gum includes up to 100 percent of the recommended daily allowance of one or more vitamins or minerals. In this manner, dental chewing gum provides, an alternative intake form for administering vitamins and minerals (e.g., as opposed to tablets or capsules). Still further, dental chewing gum may additionally or alternatively include various other herbs (e.g., herb extracts) such as ginko biloba, echinacea, kava kava or similar extracts.

FIG. 2a is a toothpick treated with a composition to deliver an herb, mineral, vitamin or similar substance to a user including anti-bacterial or antiflammatory agents such as GTE. Toothpick 201 may be derived from a natural material such as wood or from an artificial substance capable of absorbing and releasing a composition containing the agents to be delivered to the user (e.g., a paper, pulp, or similar materials).

In one embodiment, the base material is shaped in an elongated form with at least one end tapering to a point. The elongated article is immersed in a liquid treatment. The base material absorbs up to seventy five percent by weight of the treatment. In one embodiment, the base material is combined with a treatment including GTE. The treatment may be 2.5 percent by weight GTE. In another embodiment, GTE is combined with TTO in an amount sufficient to mask the odor and taste of the TTO. After saturation of the article completes, toothpick 201 is removed from the treatment and placed on a substrate to dry. Drying can be accelerated by exposing toothpick 201 to heated air or by heating the substrate. When dried, toothpick 201 may be placed in a dispenser or bundled 203 with other toothpicks (FIG. 2b). In another embodiment, the tooth pick 205 may be thin, flat and have a wedge shape with a point (FIG. 2c).

Flavoring and sweetener may optionally be added to the treatment. In one embodiment, one or more sweeteners such as saccharin, fructose, sucrose, maltose, and corn syrup may be added to the treatment. In one embodiment, mint cinnamon, bubble gum, anise or similar flavor may be added to the treatment such that the treatment is greater than 0.01% percent by weight flavorant.

The treatment may be applied by immersing the toothpick in the treatment using a liquid bath, infusing the toothpick with the treatment or spraying the toothpick with the treatment. In one embodiment, these methods of applying the treatment are engaged for a time sufficient to achieve a seventy five percent absorption of the treatment by weight.

Freeze dried GTE may be added to the treatment, commercially available from XEL Herbaceuticals of Salt Lake City, Utah. In one embodiment, TTO liquid commercially available from Natural extracts of Australia of the Los Angeles, Calif. is added to the treatment.

In one embodiment, the treatment for toothpick 201 and toothpick 201 itself are entirely formed from natural ingredients. Artificial substances are not used. The toothpick may be formed from wood such as orangewood, birch wood, basswood, or similar materials. Anise oil, peppermint oil, mint or similar naturally occurring spices and substances or combinations thereof maybe used as flavorants. In one embodiment, stevia extract or similar substance may be used as sweeteners. GTE may be used in the all natural compositions and provide anti cavity, anti-bacterial, and anti-inflammatory properties.

It is appreciated that in alternative embodiments various other treatment types for toothpicks can be formed to provide a desirable substance or substances to a user. For example, a treatment can be formed that includes one or more vitamins that are necessary for or improve nutrition, or that act as coenzymes and precursors of coenzymes in the regulation of metabolic processes. Representative vitamins include, but are not limited to, Vitamin A, beta kerotene, Vitamin B, Vitamin B6, Vitamin B12, folic acid or folate, Vitamin C, Vitamin D, Vitamin E (including tocopherols) and or tocotrienols. A treatment may additionally or alternatively include minerals such as calcium, zinc, or similar materials. For example, a treatment may include an amount of a zinc compound such as zinc gluconate or other zinc compound in an amount sufficient to inhibit symptoms associated with a common cold. In one embodiment, the treatment includes up to 100 percent of the recommended daily allowance of one or more vitamins or minerals. In this manner, treatment provides, an alternative intake form for administering vitamins and minerals (e.g., as opposed to tablets or capsules). Still further, a treatment may additionally or alternatively include various other herbs (e.g., herb extracts) such as ginko biloba, echinacea, kava kava or similar extracts.

FIG. 3 illustrates a container of mouthwash. Mouthwash 301 is used for cleansing the mouth of a user to combat organisms that cause bad breath, plaque and cavities. Mouthwash 301 is a mixture that includes a base solution and a cleansing, anti-bacterial, or anti-inflammatory agent. In one embodiment, mouthwash includes a base solution of deionized (DI) water. The base mouthwash may be combined with one or more active ingredients. The base composition and active ingredients must be in amounts that are safe for human consumption. The resulting solution is stored in clear plastic containers or similar containers for easy use.

In one embodiment, one or more base components including deionized water are combined with an active ingredient, such as an amount of GTE that is effective at reducing oral bacteria and fighting cavities. A suitable amount includes, but is not limited to, about 0.16-3.0 percent by weight GTE in a solution. In another embodiment, GTE is combined with TTO in an amount sufficient to mask the odor and taste of the TTO.

Flavoring (e.g, mint, cinnamon, bubble gum, anise, etc.), coloring, and sweeteners such as saccharin, fructose, sucrose, maltose and corn syrup may be added to the solution. The flavoring, coloring and sweeteners may be combined before being added to the solution. In one embodiment, mint, cinnamon, bubble gum, and anise or similar flavors are added to the paste, for example, in an amount of approximately five percent by weight of the paste.

The mixture may be formed by mixing one or more base components. Freeze dried GTE commercially available from XEL Herbaceauticals of Salt Lake City, Utah may be added to the solution. In another embodiment, where the solution also includes TTO, TTO liquid commercially available from Natural extracts of Australia of Los Angeles, Calif. may be added to the solution.

The following table is an exemplary composition of the paste:

| ITEM | DRY WEIGHT |
|---|---|
| D.I. water | 68.25%-71.09% |
| Glycerine USP | 25% |
| Aloe Vera | 2.0% |
| Oil of Peppermint USP | 0.3% |
| Menthol Crystal | 0.2% |
| Vitamin C | 0.27% |
| Green Tea | 0.16%-3% |
| Propionic Acid | 0.5% |
| (natural preservative) | |

In one embodiment, the mouthwash is entirely formed of natural ingredients. Artificial substances are not used. The base composition may include deionized water and glycerine. Anise oil, peppermint oil, mint or similar naturally occurring spices and substance or combinations thereof may be used as flavorants. Anise oil also has anti-bacterial properties. Chlorophyll or similar substances may be used in the composition as a natural coloring. Chlorophyll and other natural coloring substances are derived from vegetables, plants, fruits and similar natural sources. Tapioca may be used as a water binding agent and texturizing agent. In one embodiment, stevia extract or similar substances may be used as sweeteners. GTE is used in the mouthwash to provide anti-cavity, anti-bacterial and anti-inflammatory properties.

After combining the ingredients, the mouthwash is used to fill a container. The material of the container is non-porous and will not react chemically with the mouthwash. When used, the mouthwash will release active ingredients such as GTE and TTO.

It is appreciated that in alternative embodiments various other mouthwash types can be formed to provide a desirable substance or substances to a user. For example, mouthwash can be formed that includes one or more vitamins that are necessary for or improve nutrition, or that act as coenzymes and precursors of coenzymes in the regulation of metabolic processes. Representative vitamins include, but are not limited to, Vitamin A, beta kerotene, Vitamin B, Vitamin B6, Vitamin B12, folic acid or folate, Vitamin C, Vitamin D, Vitamin E (including tocopherols) and or tocotrienols. Prophylaxis paste may additionally or alternatively include minerals such as calcium, zinc, or similar materials. For example, mouthwash may include an amount of a zinc compound such as zinc gluconate or other zinc compound in an amount sufficient to inhibit symptoms associated with a common cold. In one embodiment, the paste includes up to 100 percent of the recommended daily allowance of one or more vitamins or minerals. In this manner, mouthwash provides, an alternative intake form for administering vitamins and minerals (e.g., as opposed to tablets or capsules). Still further, mouthwash may additionally or alternatively include various other herbs (e.g., herb extracts) such as ginko biloba, echinacea, kava kava or similar extracts.

FIG. 4 is an illustration of a dentifrice strip 401. In one embodiment, dentifrice strips may be made by combining one or more film forming agents with one or more active ingredients. In one embodiment, one or more film-forming agents such as pullulan, xanthan gum, locust bean gum, and carrageenan are combined with an active ingredient, such as an amount of green tea extract (GTE) that is effective at reducing oral bacteria and fighting cavities to form a base composition for the dentifrice strip. A suitable amount includes, but is not limited to, about two to five percent by weight GTE in a batch. In another embodiment, GTE is combined with tea-tree oil (TTO) in an amount sufficient to mask the odor and taste of the TTO.

Flavoring (e.g., mint, cinnamon, bubble gum, anise, etc.), coloring, and sweeteners may optionally be added to the dentifrice strip composition. In one embodiment, one or more sweeteners such as saccharin, fructose, sucrose, maltose, and corn syrup are added to the film composition. The flavoring, coloring and sweeteners may be combined before being added to the dentifrice strip composition. In another embodiment, mint flavor is added to the dentifrice strip composition, for example, in an amount of approximately 10 percent by weight of the composition.

The dentifrice strip composition may be formed by hydrating (if necessary) one or more film forming agents to create a dentifrice strip composition. To the dentifrice strip composition is added freeze-dried GTE commercially available from XEL Herbaceuticals of Salt Lake City, Utah. In an embodiment where the dentifrice strip also includes TTO, TTO liquid commercially available from Natural extracts of Australia of Los Angeles, Calif. is added to the dentifrice strip composition. The dentifrice strip composition may be heated to promote dissolution of the ingredients and subsequent drying. The film forming agents and the active ingredients must be in amounts that are non-toxic and safe for human consumption. In one embodiment, the active ingredients may be combined and optionally hydrated before being added to the dentifrice strip composition. The resulting dentifrice strip composition is then transferred to a suitable substrate where it dries to form a thin film. Drying can be accelerated by exposing the dentifrice strip composition to heated air or by heating the substrate. When dried, the dentifrice strip is cut into strips of suitable size and packaged. Representative packaging includes, but is not limited to, a spool, a single use package, and a multiple use package. The film forming agents enable the dentifrice strip composition to dry into a pliable film without interfering with or diminishing the effectiveness of the active ingredients.

In one embodiment, the dentifrice strip composition is entirely formed of natural ingredients. Artificial sweetners, colorings and similar substances are not used. In one embodiment, the composition includes sodium alginate derived from brown seaweed that functions as a thickener. Anise oil or peppermint oil, mint or similar naturally occurring spices and substances or combinations thereof are used as flavorants. Anise oil also has anti-bacterial properties. Chlorophyll or similar substances may be used in the composition as a natural coloring. Chlorophyll and other natural coloring substances are derived from vegetables, plants, fruits and similar natural sources. Tapioca starch may be used as natural water binding agent and texturizing agent. In one embodiment, stevia extract or similar substance may be used as a sweetener. Carrageenan derived from seaweed serves as a natural film forming agent. GTE is used in the all natural dentifrice strip composition and provides anti-cavity, anti-bacterial, and anti-inflammatory properties. In one embodiment, additional ingredients may include spinach extract, purified water, sorbitan acid ester and ascorbic acid. Each of these substances is derived from a naturally occurring source.

The following table is an exemplary composition of the natural ingredient composition:

| Item | Dry Weight |
|---|---|
| GTE | 0.1-8% |
| Flavoring Oil | 1-15% |
| (Peppermint/Anise) | |
| Sodium Alginate | 35-55% |
| Natural Color | 0.1-3% |
| (Chlorophyll) | |
| Tapioca Starch | 8-45% |
| Stevia Extract | 1-7% |
| Carrageenan | 4-6% |
| Glycerin | 15-35% |
| Sorbitan Acid Ester | 1-3% |

After combining the ingredients, the dentifrice strip composition is then transferred onto a suitable substrate. A suitable substrate is nonporous and will not react chemically with the film composition. As the dentifrice strip composition dries on the substrate, it changes state from a liquid to a pliable film.

The film is then cut into strips and packaged. In one embodiment, the dentifrice strip is approximately 33 mm by 26 mm in size and 20 microns thick. The film is used by consumers in the following manner. A consumer will remove a piece of film from its packaging and place the film on the front of their teeth and fold the dentifrice strip over to the back side of the teeth (FIG. 4b). The film will dissolve as it comes in contact with saliva, thereby releasing active ingredients such as GTE and TTO onto the teeth. The dentifrice strip is non toxic and may be consumed by the user without ill effects.

It is appreciated that in alternative embodiments various other dentifrice strips can be formed to provide a desirable substance or substances to a user. For example, dentifrice strips can be formed that include one or more vitamins that are necessary for or improve nutrition, or that act as coenzymes and precursors of coenzymes in the regulation of metabolic processes. Representative vitamins include, but are not limited to, Vitamin A, beta kerotene, Vitamin B, Vitamin B6, Vitamin B12, folic acid or folate, Vitamin C, Vitamin D, Vitamin E (including tocopherols) and or tocotrienols. Dentifrice strips may additionally or alternatively include minerals such as calcium, zinc, or similar materials. For example, a dentifrice strip may include an amount of a zinc compound such as zinc gluconate or other zinc compound in an amount sufficient to inhibit symptoms associated with a common cold. In one embodiment, the dentifrice strip includes up to 100 percent of the recommended daily allowance of one or more vitamins or minerals. In this manner, a dentifrice strip provider, an alternative intake form for administering vitamins and minerals (e.g., as opposed to tablets or capsules). Still further, dentifrice strips may additionally or alternatively include various other herbs (e.g., herb extracts) such as ginko biloba, echinacea, kava kava or similar extracts.

The specification has been described with reference to specific embodiments thereof. It will, however be evident that various modifications and changes can be made thereto without departing from the broader spirit and scope of the appended claims. The specification and drawings are, accordingly, to be regarded in an illustrative rather than a restrictive sense.

1. A method comprising forming a base carrier material, adding at least one of a vitamin, a mineral and an herb to the base carrier material.

2. A method comprising:
mixing a base gum composition with at least one of a vitamin, a mineral and an herb to form a chewing gum composition;
forming a chewing gum piece from the chewing gum composition.

3. The method of claim 2, wherein the base gum composition is xylitol.

4. The method of claim 2, wherein the herb is a green tea extract.

5. The method of claim 2, further comprising:
packaging the chewing gum, wherein the package is one of a spool, a single use package, a dispenser for pre-cut strips, and a blister pack.

6. The method of claim 2, further comprising:
mixing one or more natural sweeteners with the base gum composition.

7. The method of claim 6, wherein the natural sweetener is stevia extract.

8. The method of claim 2, further comprising:
mixing a natural coloring with the base gum composition.

9. The method of claim 8, wherein the natural coloring agent is chlorophyll.

10. An chewing gum produced according to the method of claim 2.

11. A method of delivering an anti-cavity and anti-inflammatory ingredient by introducing the chewing gum of claim 2 into an oral cavity.

12. A method comprising:
forming a toothpick from a base material;
saturating the toothpick with a liquid composition including one of a vitamin, an herb and a mineral.

13. The method of claim 12, wherein the base material is one of an orangewood, birchwood, and basswood.

14. The method of claim 12, wherein the herb is a green tea extract.

15. The method of claim 12, further comprising:
mixing one or more natural sweeteners with the liquid composition.

16. The method of claim 15, wherein the natural sweetener is stevia extract.

17. A toothpick produced according to the method of claim 12.

18. A method of delivering an anti-cavity and anti-inflammatory ingredient by introducing the toothpick of claim 12 into an oral cavity.

19. A method comprising:
mixing a base liquid component to form a solution;
mixing at least one of a vitamin, a mineral and an herb with the solution to form a mouthwash.

20. The method of claim 19, wherein the base liquid component is deionized water.

21. The method of claim 19, wherein the herb is a green tea extract.

22. The method of claim 19, further comprising:
mixing one or more natural flavorants with the solution.

23. The method of claim 19, wherein the natural flavorant is one of anise oil, peppermint oil and mint.

24. The method of claim 19, further comprising:
mixing a natural coloring with the solution.

25. The method of claim 24, wherein the natural coloring agent is chlorophyll.

26. A mouthwash produced according to the method of claim 19.

27. A method of delivering an anti-cavity ingredient by introducing the mouthwash of claim 19 into an oral cavity.

28. A method comprising:
mixing one or more film forming agent(s) with at least one of vitamin, a mineral and an herb to form a composition;
drying the composition on a substrate to form a strip to affix to teeth of a user.

29. The method of claim 28, wherein the one or more film forming agent(s) are one of sodium alginate and carrageenan.

30. The method of claim 28, further comprising mixing one or more natural sweeteners with the film forming agent(s).

31. The method of claim 28, further comprising:
mixing a natural coloring with the natural film forming agent(s).
